(19) United States
(12) Patent Application Publication
(10) Pub. No.: US 2023/0160918 A1
(43) Pub. Date: May 25, 2023

(54) TOOL FOR THE DISTRIBUTION OF A SAMPLE OF BIOLOGICAL OR MICROBIOLOGICAL MATERIAL AND ASSOCIATED PROCESS OF DISTRIBUTION

(71) Applicant: Copan Italia S.P.A., Brescia (IT)

(72) Inventor: Davide Dabellani, Ghedi (BS) (IT)

(21) Appl. No.: 17/919,945

(22) PCT Filed: Apr. 19, 2021
(86) PCT No.: PCT/IB2021/053208

§ 371 Date: Oct. 19, 2022

(30) Foreign Application Priority Data
Apr. 20, 2020 (IT) 102020000008416

(51) Int. Cl. G01N 35/10; B01L 3/02; B01L 3/00

(52) U.S. Cl. CPC G01N 35/1011; B01L 3/021; B01L 3/502; B01L 3/0275; B01L 2200/025; B01L 2300/0645; G01N 2035/103; B01L 2300/0848; B01L 2200/026

(57) ABSTRACT

A tool for the distribution of a sample of biological or microbiological material, the tool including an elongated body, provided with a first portion and a second portion; a distributing element, the distributing element being configured at least for distributing a sample of biological or microbiological material on a culture medium; a connector being configured for allowing the joining of the tool with a supporting element of a machine configured for carrying out a distribution of a sample of biological or microbiological material, in particular of a pipetting machine; wherein the connector is adapted and specifically configured for being removably coupled to a supporting element in alternative to another tool, in particular to a tip or pipette for the collection and/or deposit of the sample of biological or microbiological material.

### BACKGROUND OF THE INVENTION

The present disclosure refers to the field of devices for the analysis and/or processing of biological or microbiological samples, and in details concerns a tool for the distribution of a sample of biological or microbiological material. The present disclosure concerns also a process for the distribution of a sample of biological or microbiological material. The present disclosure concerns also a machine for the distribution of a sample of biological or microbiological material. The present disclosure concerns also a computer program for controlling the machine.

### Known Art

Petri dishes are dishes for culture media, on which samples of biological or microbiological materials are deposited for the purpose of cultivating cellular colonies, bacteria colonies or for the purpose of observing germinations.

The depositing of samples of biological or microbiological materials on a Petri dish takes typically place in a manual way or through an automatic pipetting module or an automatic pipetting machine. An automatic pipetting machine is an electropneumatic device suitable for collecting a predetermined amount of fluid through a pipette, or tip, and depositing this amount in a predetermined place, in particular in correspondence of a Petri dish, following an automated procedure, made repeatable by the control executed by a data processing unit. In particular, the automatic pipetting machines are configured for collecting, preferably through the actuation of a vacuum and an air pressure, a predetermined amount of liquid typically comprised between 1 μl and 1 ml. Pipetting machines of known type are configured for executing simultaneously a plurality of pipetting operations, in particular through a plurality of modules each one provided with an its own pipette, or tip.

Typically, after the depositing of a sample of biological or microbiological material on Petri dish, is executed an operation named “surface seeding” known in jargon with the name of spreading or streaking. The spreading or streaking is carried out in such a way as to disperse the sample of biological material on a determined area of the culture medium contained in the Petri dish.

According to the known technique, the spreading or streaking can manually take place through the use of a tool known as loop. This tool, comprises a first portion, or proximal portion, and a second portion or distal portion, where there is a distributing element of sample of biological or microbiological material. The tool herein described has a substantially elongated shape and develops substantially along an axial direction. The tool comprises also a rod, which is interposed between the first portion, or proximal portion, and the second portion, or distal portion.

The spreading or streaking of a sample of biological or microbiological material through manual technique is characterized by some drawbacks. The spreading or streaking of a sample of biological or microbiological material is a process sensitive to the pressure that the tool exercises on the culture medium. In particular, when the pressure exercised by the distributing element during the spreading or streaking is too low, there is the risk not to distribute in a sufficiently uniform way the sample of biological or microbiological material on the determined area of the culture medium. Vice versa, when the pressure exercised by the distributing element during the spreading or streaking is too high, there is the risk to incise the medium and to distort the future growth of bacteria.

Even when the activity of spreading or streaking has to be carried out through a machine, this activity would comprise the need to use two different machines: a pipetting machine, and a machine for the execution of the spreading or streaking of the sample. This is expensive and causes manipulations of the sample which dilate treatment times of samples, in particular when there is the need of executing a big quantity of operations of spreading or streaking.

### Purposes of the Invention

It is a purpose of the present disclosure to describe a tool for the distribution of a sample of biological or microbiological material which allows to solve the above-described drawbacks. It is in particular a purpose of the present disclosure to describe a tool for the distribution of a sample of biological or microbiological material which allows to obtain a distribution of sample of biological or microbiological material on the culture medium in a uniform way and/or with a uniform pressure and/or sufficient to ensure the correct distribution without the risk of incision of the culture medium itself.

More in details, it is a purpose of the present disclosure to describe a tool for the distribution of a sample of biological or microbiological material which can be effectively coupled to a portion or module of a robot, or of a machine for the distribution of a sample of biological or microbiological material and containing the costs of the automatic plant in its entirety.

It is another purpose of the present disclosure to describe a tool for the distribution of a sample of biological or microbiological material which allows to collect a predetermined portion of a sample of biological or microbiological material.

It is also purpose of the present disclosure to describe a process of distribution of a sample of biological or microbiological material on a culture medium which is free from the above-described drawbacks.

It is also purpose of the present disclosure to describe a machine, in particular a pipetting machine, which is configured for allowing also the distribution of a sample of biological or microbiological material, in such a way as to mitigate and/or solve the above-described drawbacks.

It is also purpose of the present disclosure to describe a computer program adapted to allow the movement of a tool for the distribution of a sample of biological or microbiological material and/or of robot or machine for the distribution of a sample of biological or microbiological material which is free from the above-described drawbacks.

### SUMMARY

These and other purposes are obtained through a tool for the distribution of a sample of biological or microbiological material according to one or more of the following aspects, which can be combined among them.

According to an aspect, it is described a tool (10) for the distribution of a sample of biological or microbiological material, the tool (10) comprising:
an elongated body (13), provided with a first portion (11), or proximal portion, and a second portion (12), or distal portion, different from the first portion (11);
a distributing element (14), positioned in correspondence of the second portion (12), the distributing element (14) being configured at least for distributing a sample of biological or microbiological material on a culture medium;
a connector (15), positioned in correspondence of the first portion (11), the connector (15) being configured for allowing the removable joining of the tool (10) with a supporting element (20) of a machine (40) configured for carrying out a distribution of a sample of biological or microbiological material, in particular of a pipetting machine (40) configured for carrying out a distribution of a sample of biological or microbiological material;
   wherein the distributing element (14) is electrically and/or capacitively coupled with the elongated body (13) and/or with the connector (15); the tool (10) being configured for acting, in use, as capacitive probe adapted to allow the detection of a contact at least of the distributing element (14) with a substance, in particular with the culture medium and/or with the sample of biological or microbiological material and for allowing to keep said contact during the movement of the tool for the distribution of the sample of biological or microbiological material.

According to another non-limiting aspect, the connector (15) is adapted to and specifically configured for being removably coupled to the supporting element (20) of the machine (40).

According to another non-limiting aspect, the machine (40) is configured for automatically carrying out at least a step of depositing of a sample of biological or microbiological material in the container (30) through a release of the sample of biological or microbiological material from a tip or pipette removably coupled to the supporting element (20) in alternative to the tool (10).

According to another non-limiting aspect, the tool (10) is configured for being removably coupled to the supporting element (20) in alternative to another tool, in particular to a tip or pipette for collecting and/or depositing the sample of biological or microbiological material.

According to another non-limiting aspect, the tip or pipette is configured for at least temporarily holding a sample of biological or microbiological material, in particular in liquid or fluid form.

According to an aspect, it is described a tool (10) for the distribution of a sample of biological or microbiological material, the tool (10) comprising:

an elongated body (13), provided with a first portion (11), or proximal portion, and a second portion (12), or distal portion, different from the first portion (11);

a distributing element (14), positioned in correspondence of the second portion (12), the distributing element (14) being configured at least for distributing a sample of biological or microbiological material on a culture medium;

a connector (15), positioned in correspondence of the first portion (11), the connector (15) being configured for, and/or specifically designed to, allowing the joining of the tool (10) with a supporting element (20) of a machine (40) configured for carrying out a distribution of a sample of biological or microbiological material, in particular of a pipetting machine (40) configured for carrying out a distribution of a sample of biological or microbiological material;

wherein the connector (15) is adapted and specifically configured for being removably coupled to a supporting element (20) in alternative to another tool, in particular to a tip or pipette for collecting and/or depositing the sample of biological or microbiological material.

According to another non-limiting aspect, the distributing element (14) is electrically and/or capacitively coupled to the elongated body (13) and/or to the connector (15); the tool (10) being configured for acting, in use, as capacitive probe adapted to allow the detection of a contact at least of the distributing element (14) with a substance, in particular with the culture medium and/or with the sample of biological or microbiological material.

According to another non-limiting aspect, the tool (10) is configured for allowing to keep said contact during the movement of the tool for the distribution of the sample of biological or microbiological material.

According to another non-limiting aspect, the pipetting machine (40) is an automatic pipetting machine.

According to another non-limiting aspect, at least part of the elongated body (13), in particular the first portion (11), is introduced within the connector (15) and presents a contact portion (13s) operatively accessible by an electrode (20a) positioned on the supporting element (20) of the machine (40).

According to another non-limiting aspect, the contact portion (13s) is an electrically conductive portion.

According to another non-limiting aspect, the contact portion (13s) is configured for allowing in use the detection of a difference of capacity, for allowing the transmission of an electrical signal of capacity variation to the electrode (20a), at the contact at least of the distributing element (14) with the substance, in particular with the culture medium and/or with the sample of biological or microbiological material.

According to another non-limiting aspect, the contact portion (13s) is integral with the elongated body (13).

According to another non-limiting aspect, the contact portion (13s) is in electrical contact with the elongated body (13).

According to another non-limiting aspect, the elongated body (13) is realized in electrically conductive material.

According to another non-limiting aspect, the connector (15) comprises an engaging portion (16) configured for allowing the coupling of the connector (15) with the supporting element (20).

According to another non-limiting aspect, the engaging portion (16) comprises a recess suitable for housing at least part of the supporting element (20).

According to another non-limiting aspect, the contact portion (13s) is realized in correspondence of the engaging portion (16), in particular within the recess of the engaging portion (16).

According to another non-limiting aspect, the contact portion (13s) is substantially discoidal-shaped and is positioned in substantial correspondence of a bottom wall (16f) present in the recess of the engaging portion (16).

According to another non-limiting aspect, the contact portion (13s) covers at least part of the bottom wall (16f), optionally substantially all the bottom wall (16f).

According to another non-limiting aspect, the contact portion (13s) is realized in correspondence of a side wall (16l) of the recess.

According to another non-limiting aspect, the contact portion (13s) in particular comprises at least part of the side wall (16l), optionally the entire side wall (16l).

According to another non-limiting aspect, the side wall (16l) is configured for allowing the realization of an electrical contact with an electrode (20a) of the supporting element (20) when said supporting element (20) is introduced in the recess and is inserted with mechanical interference in the recess itself.

According to another non-limiting aspect, the connector (15) comprises an engaging portion (16) configured for allowing the coupling with the supporting element (20), optionally wherein the engaging portion (16) comprises a protrusion designed to be at least partially introduced within the supporting element (20).

According to another non-limiting aspect, the connector (15) presents a guiding cavity (17) designed to house, and in particular to hold, at least part of the elongated body (13), and in particular wherein the guiding cavity (17) is designed to house, and in particular to hold, at least part of the first portion (11) or proximal portion of the elongated body (13).

According to another non-limiting aspect, the elongated body (13) substantially and/or mainly extends along an axial direction (X).

According to another non-limiting aspect, the elongated body (13) is at least partially flexible, optionally integrally flexible.

According to another non-limiting aspect, the connector (15) is fixed in unremovable and/or substantially rigid way to the elongated body (13).

According to another non-limiting aspect, the elongated body (13) presents at least a central portion, interposed between the first portion (11) or proximal portion and the second portion (12) or distal portion, substantially cylindrical-shaped.

According to another non-limiting aspect, the guiding cavity (17) communicates with the engaging portion (16), in particular with the recess.

According to another non-limiting aspect, the guiding cavity (17) is designed to allow to house at least part of the elongated body (13) through an introduction with mechanical contrast and/or through a sliding friction developing, at the introduction, between an inner side surface of the guiding cavity (17) and an outer side surface of the part of elongated body (13) here introduced; said mechanical contrast and/or said sliding friction being such that the elongated body (13), when introduced within the guiding cavity (17) remains held there by action of the mechanical contrast and/or sliding friction.

According to another non-limiting aspect, the engaging portion (16) presents:

at least a side wall (16l) and

a bottom wall (16f),

and wherein the bottom wall (16f) is at least partially flat.

According to another non-limiting aspect, the first portion (11) or proximal portion of the elongated body (13) ends in correspondence of a junction zone between the guiding cavity (17) and the bottom wall (16f) and comprises a contact portion (13s), designed to realize an electrical contact portion with said electrode (20a) having a size sufficient to transmit an electrical signal of capacity variation, when, in use, the tool (10) enters in contact with the culture medium and/or with a sample of biological or microbiological material.

According to another non-limiting aspect, the contact portion (13s) is configured for realizing an electrical continuity between the elongated body (13) and the connector (15).

According to another non-limiting aspect, the connector (15) is at least partially electrically conductive and/or is in electrical contact with the elongated body (13).

According to another non-limiting aspect, the connector (15) is configured for allowing the transmission of an electrical signal of capacity variation to the electrode (20a).

According to another non-limiting aspect, the connector (15) comprises a body of substantially asymmetric shape along an axis coincident with the axis (X) along which the elongated body (13) mainly develops.

According to another non-limiting aspect, the distributing element (14) presents an asymmetric shape.

According to another non-limiting aspect, the distributing element (14) is integral with the elongated body (13).

According to another non-limiting aspect, the distributing element (14) comprises an electrically and/or capacitively conductive substantially tubular portion; the substantially tubular portion being folded in such a way to misalign itself from the axis (X) along which the elongated body (13) substantially develops.

According to another non-limiting aspect, the tubular portion substantially comprises a same transverse size, in particular a same diameter, of the transverse size, in particular of the diameter, of the elongated body (13).

According to another non-limiting aspect, the distributing element (14) is folded backwards according to a curvilinear conformation and/or so that one end thereof is at a height (Qi) positioned between two end heights between which the tool (10) is enclosed, a first end height (Qe) thereof is a height reached at the end by the second portion (12).

According to another non-limiting aspect, the distributing element (14) comprises an end portion joined, optionally welded, to the elongated body (13), said end portion realizing a substantially closed ring.

According to another non-limiting aspect, the distributing element (14) presents a substantially hook shape.

According to another non-limiting aspect, the distributing element (14) comprises a first end portion directly joined to the elongated body (13) and a second end portion, different from the first end portion and juxtaposed to the first end portion; the second end portion ending in position proximal to the elongated body (13) or being rigidly joined, optionally welded, to the elongated body (13).

According to another non-limiting aspect, the substantially tubular portion is folded backwards so that one end thereof is at a more inner height with respect to an end height (Qe) reached by the second portion (12) or distal portion.

According to another non-limiting aspect, the distributing element (14) comprises a substantially spheroidal or at least partially spheroidal body, in particular a substantially spheroidal or at least partially spheroidal body whose diameter is higher with respect to a thickness and/or a transversal size that the elongated body (13) assumes along a direction substantially orthogonal to the axis (X) along which the elongated body (13) substantially develops.

According to another non-limiting aspect, the distributing element (14) is substantially “L”-shaped.

According to another non-limiting aspect, the distributing element (14) comprises a first portion aligned along the axis (X) and/or extending uninterruptedly from the elongated body (13) and a second portion joined and orthogonal to the first portion.

According to another non-limiting aspect, the distributing element (14) comprises a hollow body, which detects a cavity (14c) adapted at least to contain a predetermined amount of substance, in particular a predetermined amount of fluid or liquid and/or sample of biological or microbiological material and/or a predetermined amount of culture medium.

According to another non-limiting aspect, the cavity (14c) is adapted to transfer a sample of biological or microbiological material.

According to another non-limiting aspect, the cavity (14c) has a circular section, or a rectangular or square section, and has an its own axis (K) oriented in transversal direction, optionally substantially orthogonal, to the axis (X) along which the elongated body (13) substantially develops.

According to another non-limiting aspect, the cavity (14c) is adapted to contain a volume of substance at least equal to 0.5 μl, or 1 μl, or 2 μl, or 5 μl, or 10 μl, or 30 μl.

According to another non-limiting aspect, the cavity (14c) is adapted to contain a predetermined amount of fluid or liquid by surface tension.

According to another non-limiting aspect, the tool (10) is a single use tool. Alternatively, according to another non-limiting aspect, the tool (10) is a tool adapted to, and/or specifically designed to, be used a plurality of times, and in particular is a sterilizable tool (10).

According to another non-limiting aspect, at least the elongated body (13) is realized at least partially in metallic material.

According to another non-limiting aspect, the metallic material is a low thermal conductivity metallic material.

According to another non-limiting aspect, the metallic material comprises a Nickel-Chromium alloy, in particular with amount of Nickel equal to or higher than 60% in weight, more preferably equal to or higher than 63% in weight and even more preferably equal to or higher than 65% in weight.

According to another non-limiting aspect, the Nickel-Chromium alloy comprises a UNS N06082 or Inconel 82 alloy, and/or is an alloy comprising Manganese, Niobium and, optionally, Iron.

According to another non-limiting aspect, in said alloy, Iron is contained in amount comprised between 0 and 3% in weight.

According to another non-limiting aspect, at least the elongated body (13) is realized at least partially in plastic material.

According to another non-limiting aspect, the distribution of a sample of biological or microbiological material comprises, optionally is, a spreading or streaking of a sample of biological or microbiological material.

According to another aspect, it is described a use of the tool (10) according to one or more of these aspects, for the distribution of a sample of biological or microbiological material within a container (30) comprising or constituted by a Petri dish.

According to another non-limiting aspect, it is described a use of the tool (10) according to one or more of these aspects, in association to a pipetting machine (40) and/or in association to a machine (40) for the distribution of samples of biological or microbiological material.

According to an aspect, according to the present disclosure, it is described a spreading or streaking of a sample of biological or microbiological material by means of a tool (10) according to one or more of these aspects, wherein said spreading or streaking is realized within a container (30), in particular a container for samples of biological or microbiological material.

According to another non-limiting aspect, the collection of said sample of biological or microbiological material is carried out through a machine (40), in particular a pipetting machine configured for housing, in particular on a supporting element (20), the tool (10) in alternative to a tip or pipette configured for being removably fixed to the machine (40).

According to another aspect, it is described a process of distribution of a sample of biological or microbiological material on a culture medium, the process comprising:

a step (1000) of depositing of a sample of biological or microbiological material, within a container (30);
a subsequent and first step of movement (1004) of a tool (10) for the distribution of a sample of biological or microbiological material according to one or more of the aspects herein described;

wherein through the first step of movement (1004) of the tool (10), the distributing element (14) is moved between a first position, wherein the distributing element (14) is in a remote position with respect to the container (30), and a second position, wherein the distributing element (14) is in a position nearer to the container (30);

a step of electric or electronic measurement of a capacity (1005), wherein said capacity is detected through the tool (10), and wherein the step of measurement of the capacity (1005) is carried out at least partially simultaneously with the first step of movement (1004) of the tool (10),

wherein, when a variation of capacity is measured, the movement of the tool (10) in the first step of movement (1004) is at least temporarily stopped and/or is finished and the distributing element (14) is in substantial contact with the sample of biological or microbiological material and/or with a predetermined amount of culture medium,

and wherein after the first step of movement (1004) is stopped, a step of distribution of the sample of biological or microbiological material by means of the tool (10) takes place.

According to another non-limiting aspect, at least the first step of movement (1004) and/or the step of electric or electronic measurement of a capacity (1005) and/or the step of distribution of the sample of biological or microbiological material are carried out automatically in a predefined sequence and/or are carried out through a machine (40) configured for carrying out a distribution of a sample of biological or microbiological material, preferably a pipetting machine (40) configured for carrying out a distribution of a sample of biological or microbiological material.

According to another non-limiting aspect, after the first step of movement (1004) is stopped, the process comprises a step of introduction, wherein the tool (10) is at least partially introduced within the sample of biological or microbiological material and/or in the culture medium contained in the container (30).

According to another non-limiting aspect, when a variation of capacity is measured, the movement of the tool (10) in the first step of movement is kept for a time sufficient to introduce at least partially the tool (10) within the sample of biological or microbiological material and/or in the culture medium contained in the container (30).

According to another non-limiting aspect, the tool (10) is introduced within the sample of biological or microbiological material and/or in the culture medium for a depth comprised between 1/10 mm and 1 mm.

According to another non-limiting aspect, the process comprises a step of movable coupling (1003), wherein a connector (15) of the tool (10), is removably coupled to a supporting element (20) of a machine (40) configured for carrying out a distribution of a sample of biological or microbiological material, optionally a pipetting machine (40) configured for carrying out a distribution of a sample of biological or microbiological material; said step of movable coupling (1003) taking place at least before said first step of movement (1004).

According to another non-limiting aspect, the process comprises a step of replacement of the tip or pipette of a pipetting machine (40) with a tool (10) for the distribution of a sample of biological or microbiological material according to one or more of the aspects herein described, wherein the tool (10) is coupled to the pipetting machine (40) instead of the tip or pipette.

According to another non-limiting aspect, the step of distribution is a step of distribution of the sample of biological or microbiological material on the culture medium through the tool (10) moved by the pipetting machine (40).

According to another non-limiting aspect, the step of replacement of the tip or pipette comprises and/or is followed by the step of movable coupling (1003).

According to another aspect, it is described a process of distribution of a sample of biological or microbiological material on a culture medium, the process comprising:

a step (1000) of depositing of a sample of biological or microbiological material, within a container (30), the depositing taking place through a pipetting machine (40);
a subsequent and first step of movement (1004) of a tool (10) for the distribution of a sample of biological or microbiological material according to one or more of the aspects herein described;

wherein through the first step of movement (1004) of the tool (10), the distributing element (14) is moved between a first position, wherein the distributing element (14) is in a remote position with respect to the container (30), and a second position, wherein the distributing element (14) is in a position closer to the container (30);

the process comprising a step of movable coupling (1003), wherein a connector (15) of the tool (10), is removably coupled to a supporting element (20) of the pipetting machine (40) configured for carrying out a distribution of a sample of biological or microbiological material, in particular of the pipetting machine (40) configured for carrying out a distribution of a sample of biological or microbiological material; said step of movable coupling (1003) taking place at least before said first step of movement (1004);

and wherein after the first step of movement (1004) takes place a step of distribution of the sample of biological or microbiological material by means of the tool (10).

According to another aspect, it is herein described a process of distribution of a sample of biological or microbiological material on a culture medium, the process comprising:

a step (1000) of depositing of a sample of biological or microbiological material, through a tip or pipette of a pipetting machine (40), within a container (30) provided with a culture medium;
a step of replacement of the tip or pipette of the pipetting machine (40) with a tool (10) for the distribution of a sample of biological or microbiological material according to one or more of the aspects herein described, wherein the tool (10) is coupled to the pipetting machine (40) instead of the tip or pipette;
a step of distribution of the sample of biological or microbiological material on the culture medium through the tool (10) moved by the pipetting machine (40).

According to another non-limiting aspect, the pipetting machine (40) is a machine configured for carrying out also a distribution of a sample of biological or microbiological material.

According to another non-limiting aspect, the step of replacement of the tip or pipette of the pipetting machine (40) with the tool (10) comprises a step of movable coupling (1003), wherein a connector (15) of the tool (10), is removably coupled to the supporting element (20) of the pipetting machine (40); and wherein the process comprises also a first step of movement (1004) of the tool (10) wherein the distributing element (14) is moved between a first position wherein the distributing element (14) has been coupled to the pipetting machine (40) and is in a remote position with respect to the container (30), and a second position, wherein the distributing element (14) is in a position closer to the container (30) and in contact at least with the sample of biological or microbiological material.

According to another non-limiting aspect, after the first step of movement (1004) takes place the step of distribution of the sample of biological or microbiological material by means of the tool (10).

According to another non-limiting aspect, the capacity is an electrical capacity.

According to another non-limiting aspect, the process comprises a step of measurement of a capacity (1005), wherein said capacity is detected through the tool (10), and wherein the step of measurement of the capacity (1005) is carried out at least partially simultaneously with the first step of movement (1004) of the tool (10).

According to another non-limiting aspect, when a variation of capacity is measured indicating a contact, the movement of the tool (10) in the first step of movement (1004) is at least temporarily stopped and/or is finished and the distributing element (14) is in substantial contact with the sample of biological or microbiological material.

According to another non-limiting aspect, when a variation of capacity is measured indicating a contact, the first step of movement (1004) is stopped and the step of distribution of the sample of biological or microbiological material takes place.

According to another non-limiting aspect, after the detection of a variation of capacity indicating a contact between the distributing element (14) and the sample of biological or microbiological material, the first step of movement (1004) is stopped and the step of distribution of the sample takes place.

According to another non-limiting aspect, in the first step of movement (1004) the container (30) is kept in a predetermined position, in particular in a fixed position.

According to another non-limiting aspect, in the second position, the distributing element (14) is in substantial contact with at least part of the sample of biological or microbiological material contained in the container (30).

According to another non-limiting aspect, the step of movable coupling (1003) comprises an electric and/or capacitive coupling realized between an electrode (20a) positioned on the supporting element (20) and a contact portion (13s) of the elongated body (13).

According to another non-limiting aspect, the contact portion (13s) lays in correspondence of a side wall (16l) of the engaging portion (16), and the electric coupling is realized between the electrode (20a) and the contact portion (13s) introducing the supporting element (20) at least partially within the engaging portion (16) such that a sliding friction and a contrast force on the side wall (16l) of the engaging portion (16) itself are exercised.

According to another non-limiting aspect, the electric and/or capacitive coupling is such that, through the electrode (20a), the machine (40) can detect when the distributing element (14) enters in substantial contact at least with the sample of biological or microbiological material and/or with the culture medium.

According to another non-limiting aspect, the step of distribution of the sample of biological or microbiological material by means of the tool (10) comprises a second step of movement (1006), wherein a relative movement between the distributing element (14) and the container (30) takes place such that, in the continuation of the relative movement, the distributing element (14) remains in substantial contact with the sample of biological or microbiological material and/or with the predetermined amount of culture medium contained in the container (30) and wherein, through the relative movement, the distributing element (14) traces a predetermined trajectory on the area enclosed by the container (30).

According to another non-limiting aspect, in the continuation of the relative movement, the distributing element (14) is at least partially introduced in the sample of biological or microbiological material and/or in the predetermined amount of culture medium.

According to another non-limiting aspect, the relative movement between the distributing element (14) and the container (30) allows for realizing a personalized and/or individualized pattern of distribution of the sample of biological or microbiological material.

According to another non-limiting aspect, the step of distribution of the sample of biological or microbiological material by means of the tool (10), or the second step of movement (1006), comprises a rotation of the container (30) around an axis (X′) substantially parallel to the axis (X) along which the elongated body (13) substantially develops.

According to another non-limiting aspect, the step of distribution of the sample of biological or microbiological material by means of the tool (10), or the second step of movement (1006), comprises a translation of the elongated body (13) along a plane comprising the axis (X) along which the elongated body (13) substantially develops and/or comprises a translation of the body (13) with respect to the area enclosed by the container (30).

According to another non-limiting aspect, in the step of distribution of the sample of biological or microbiological material by means of the tool (10), or in the second step of movement (1006), the rotation of the container (30) and the translation of the elongated body (13) are carried out at least partially simultaneously.

According to another non-limiting aspect, in the step of distribution of the sample of biological or microbiological material by means of the tool (10), or in the second step of movement (1006), the rotation of the container (30) and the translation of the elongated body (13) are carried out in alternated way.

According to another non-limiting aspect, the rotation of the container (30) takes place by holding the container (30) by means of a plurality of arms (41) adapted to surround at least part of a side wall of the container (30) itself and/or by means of a device adapted to create a differential of pressure with a bottom wall of the container (30).

According to another non-limiting aspect, the device adapted to create a differential of pressure with the bottom wall of the container (30) comprises a suction cup.

According to another non-limiting aspect, the plurality of arms (41) and/or the device adapted to create a differential of pressure is mechanically connected with a motor configured for allowing a controlled rotation thereof.

According to another non-limiting aspect, the container (30) is a container configured for containing samples of biological or microbiological material, the container (30) preferably comprising a Petri dish.

According to another non-limiting aspect, the step (1000) of depositing of the sample of biological or microbiological material within the container (30) is carried out through the machine (40), which is adapted to hold at least temporarily a predetermined volume of the substance and to transfer said predetermined volume of the sample of biological or microbiological material within said container (30).

According to another non-limiting aspect, the step of movable coupling (1003) comprises an axial approximation of the distal end of the supporting element (20) with the connector (15), in particular with the engaging portion (16) of the connector (15).

According to another non-limiting aspect, the step of movable coupling (1003) comprises a subsequent introduction of the supporting element (20) at least partially into the engaging portion (16) such that a sliding friction and a contrast force on the truncated cone-shaped side wall (16l) of the engaging portion (16) itself are exercised, said sliding friction and/or said contrast force being sufficient to hold the tool (10) on the supporting element (20), in particular at least during a step of uplifting of the tool (10) itself.

According to another non-limiting aspect, as the introduction of the supporting element (20) increases into the engaging portion (16), due to a conicity assumed by the side wall (16l), said sliding friction and/or said contrast force increases.

According to another non-limiting aspect, the process comprises a step (1009) of depositing of a culture medium within the container (30) and the step (1000) of depositing of the sample of biological or microbiological material takes place after the depositing of the culture medium.

According to another non-limiting aspect, the process comprises a step of collecting (1002) of said tool (10) from a loader (50), or tray, adapted to contain a plurality of tools (10) and comprising a plurality of seats (51) adapted to define a predetermined spatial configuration for the plurality of tools (10).

According to another non-limiting aspect, in the step of collecting (1002) is executed said step of movable coupling (1003), from which derives that between the supporting element (20) and the connector (15) or between the supporting element (20) and the engaging portion (16) of connector (15) it is realized a mechanical contrast and/or a sliding friction such as to allow the uplifting of the tool (10) from the respective seat.

According to another non-limiting aspect, by effect of the step of collecting (1002) and/or of the step of movable coupling (1003), through the connector (15) the tool (10) transmits an electrical signal of capacitive variation towards an electrode (20a) of the supporting element (20).

According to another non-limiting aspect, the process comprises a procedure of high temperature sterilization of the tool (10), preferably by radiation or by means of flame heating.

According to another non-limiting aspect, the procedure of sterilization comprises the heating at least of the elongated body (13) and/or of the distributing element (14) at a temperature up to 800° C.

According to the present disclosure, it is herein described a pipetting machine (40), configured for carrying out one or more steps of the process according to one or more aspects of the present disclosure.

According to the present disclosure, it is herein described a computer program, stored on a memory support; said computer program being adapted to be executed by at least an electronic computer and comprising portions of software code which when executed cause the execution of one or more steps of the process according to one or more of these aspects.

According to the present disclosure, it is described a computer program, stored on a memory support; said computer program being adapted to be executed by at least an electronic computer and comprising portions of software code which when executed cause the execution of:

a first step of movement (1004) of a movable supporting element (20) of a pipetting machine (40), the supporting element (20) being configured for being removably connected with a tool (10) for the distribution of a sample of biological or microbiological material according to one or more of the aspects herein described, wherein through the first step of movement (1004) of the supporting element (20), a distributing element (14) of the tool (10) for the distribution of a sample of biological or microbiological material is moved between:
a first position, wherein the distributing element (14) is in a remote position with respect to a container (30) adapted to contain, in use, a sample of biological or microbiological material and a predetermined amount of culture medium, and
a second position, wherein the distributing element (14) is in a position at least closer to the container (30);

a step of electric or electronic measurement of a capacity (1005) detected through the tool (10), wherein in the step of measurement of the capacity (1005), carried out at least partially simultaneously with the first step of movement (1004) of the tool (10), the pipetting machine (40) carries out a measurement of a capacity through an electric and/or capacitive contact with an electrically conductive portion of the tool (10) and wherein, when said pipetting machine (40) measures a variation of capacity, the movement of the supporting element (20) in the first step of movement (1004) is at least temporarily stopped so that the distributing element (14) is in substantial contact with the sample of biological or microbiological material and/or with the predetermined amount of culture medium,

a step of distribution of the sample of biological or microbiological material by means of the tool (10), carried out after the step of measurement of the capacity (1005).

According to another non-limiting aspect, the step of distribution of the sample of biological or microbiological material by means of the tool (10) comprises a second step of movement (1006), wherein the portions of software code, when executed, cause a relative movement between the distributing element (14) and the container (30) such that, during the relative movement, the distributing element (14) remains in substantial contact with the sample of biological or microbiological material and/or with the predetermined amount of culture medium contained in the container (30) and wherein, through the relative movement, the distributing element (14) traces a predetermined trajectory on the area enclosed by the container (30).

According to another non-limiting aspect, the pipetting machine (40) carries out the measurement of said capacity through a capacitive sensor installed on said machine (40) and/or on the supporting element (20).

According to another non-limiting aspect, the pipetting machine (40) carries out a measurement of capacity through an electrical and/or capacitive contact realized between an electrically conductive portion of the tool (10) and at least part of the supporting element (20).

According to another non-limiting aspect, the portions of software code when executed, cause the execution of a step (1000) of depositing of a substance, in particular of a predetermined amount of fluid or liquid and/or sample of biological or microbiological material, wherein the pipetting machine (40) causes the release of a predefined amount, in particular a predefined volume, of a sample of biological or microbiological material, within the container (30).

According to another non-limiting aspect, the portions of software code cause, when executed, the execution of the step (1000) of depositing of a sample of biological or microbiological material before the execution of said first step of movement (1004).

According to another non-limiting aspect, in the step (1000) of depositing of the sample of biological or microbiological material, the portions of software code cause the actuation of a system of removal of a vacuum previously created within a tip or pipette removably coupled to the supporting element (20) for the execution of the step (1000) of depositing and/or cause the activation of a system of injection for the injection of air within a tip or pipette removably coupled to the supporting element (20) for the execution of the step (1000) of depositing.

According to another non-limiting aspect, the portions of software code, when executed, cause the execution of a step of movable coupling (1003) wherein the machine (40) is actuated for carrying out a step of axial approximation of a distal end of the supporting element (20) with a connector (15) of the tool (10), the step of movable coupling (1003) being executed before the first step of movement (1004).

According to another non-limiting aspect, in the step of movable coupling (1003) the portions of software code, when executed, cause the movement of the supporting element (20) in such a way that an introduction of the supporting element (20) is realized at least partially within an engaging portion (16) of the connector (15) such that a sliding friction and a contrast force on the truncated cone-shaped side wall (16l) of the engaging portion (16) itself are exercised, said sliding friction and/or said contrast force being sufficient to hold the tool (10) on the supporting element (20), in particular at least during a step of uplifting of the tool (10) itself.

According to another non-limiting aspect, the step of movable coupling (1003) comprises a movement of the supporting element (20) along a horizontal plane, in such a way that it is positioned in substantial axial correspondence with an engaging portion (16) of said connector.

According to the present disclosure it is described a computer program, stored on a memory support; said computer program being adapted to be executed by at least an electronic computer and comprising portions of software code which when executed cause the execution of:

an actuation of a pipetting machine (40) for the execution of a step (1000) of depositing of a sample of biological or microbiological material, through a tip or pipette of a pipetting machine, within a container (30) provided with a culture medium;
an actuation of the pipetting machine (40) for the execution of a step of replacement of the tip or pipette with a tool (10) for the distribution of a sample of biological or microbiological material according to one or more of present aspects, wherein the tool (10) is coupled to the pipetting machine (40) instead of the tip or pipette;
an actuation of the pipetting machine (40) for the execution of a step of distribution of the sample of biological or microbiological material on the culture medium through the tool (10) moved by the pipetting machine (40).

### DRAWINGS

The object of the present disclosure will be now described in some preferred and non-limiting embodiments, through the help of drawings wherein:

FIG. 1 shows a perspective view of a tool according to the present disclosure;

FIG. 2 shows a partial sectional view of the tool of FIG. 1;

FIG. 3 shows a first configuration of use wherein the tool object of the present disclosure is not in contact with a substance;

FIG. 4 shows a second configuration of use wherein the tool object of the present disclosure is in contact with a substance, showing an alteration of the capacity;

FIG. 5 shows an embodiment of a distributing element of the tool object of the present disclosure;

FIG. 6 shows a perspective view of another embodiment of a distributing element of the tool object of the present disclosure;

FIG. 7 shows a sectional view, along the lines A-A of FIG. 6, of the embodiment of FIG. 6;

FIG. 8 shows a section of a connector of the tool object of the present disclosure;

FIG. 9 shows a perspective view of a specific embodiment of the tool object of the present disclosure;

FIG. 10 shows a side view of a portion of a machine for the distribution of a sample of biological or microbiological material;

FIG. 11 shows a perspective view, wherein a portion of a machine for the distribution of a sample of biological or microbiological material collects a tool according to the present disclosure from a support;

FIG. 12 shows a block diagram with a plurality of steps carried out by a process of distribution of a sample of biological or microbiological material according to the present disclosure;

FIG. 13, FIG. 14 and FIG. 15 show steps in sequence of movement of a tool object of the present disclosure with respect to a container for samples of biological or microbiological material.

### DETAILED DESCRIPTION

With the reference number 10 is indicated in its entirety a tool for the distribution of a sample of biological or microbiological material. As it will be clear in the light of the following description, the tool 10 is configured for being manipulated by a machine, in particular by a machine for the distribution of a sample of biological or microbiological material, and/or by a pipetting machine; said machine, which in the following description will be indicated with the numeric reference 40, is configured for operating on at least a container 30, optionally on a plurality of containers 30 in such a way that, through the tool 10, a sample of biological or microbiological material can be distributed on a predetermined area of the container 30, without incurring in the previously identified drawbacks and allowing a very precise distribution of said sample, in particular by avoiding to incise the culture medium contained in the container itself. Although in principle the container 30 can be any container adapted to contain a solid and/or fluid substance, in a non-limiting but preferred embodiment, the container 30 is a container specifically configured for containing samples of biological or microbiological material, and/or is a container adapted to contain a culture medium. A culture medium is a solid or liquid solution comprising substances on which it is possible to grow cells, including the ones of bacteria. In a non-limiting embodiment, the container 30 is, or comprises, a Petri dish, and can comprise a bottom wall, upon which it is deposited the culture medium, and at least a side wall (or more side walls, if the plant of the container is not curved), for the containing of the culture medium and/or of the sample, which departs preferably orthogonally from the bottom wall.

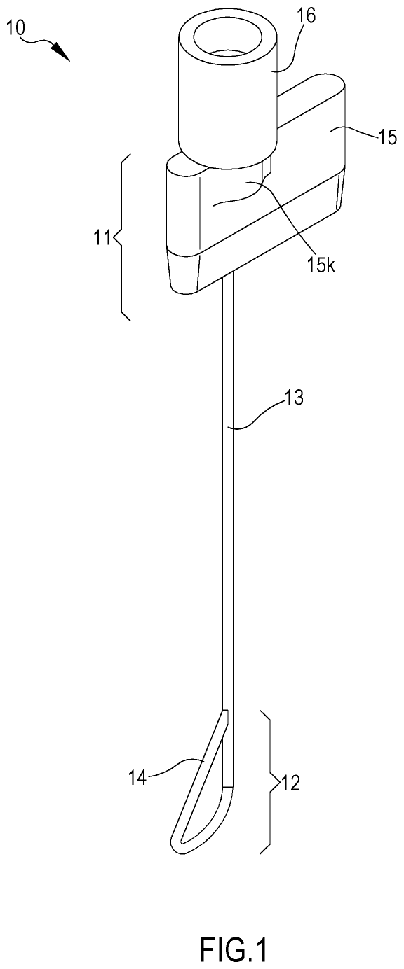

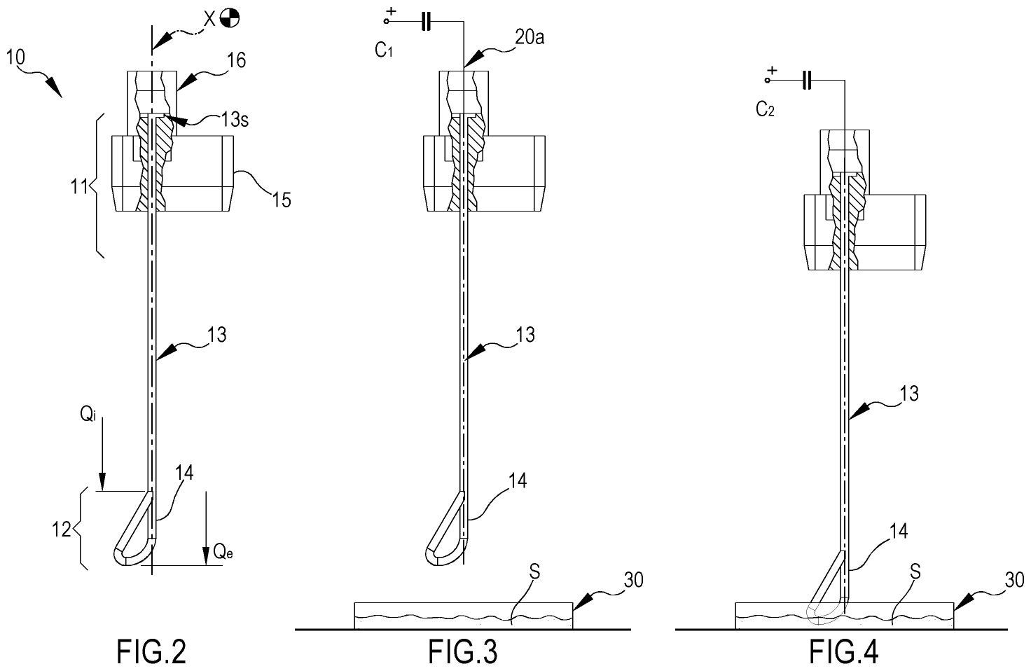

As shown in FIG. 1 and in FIG. 2, the tool 10 comprises an elongated body 13 which develops primarily along an axis which is here identified as axis X. The elongated body 13 detects a first portion 11, or proximal portion, and a second portion 12, or distal portion, different and in particular opposed to the first portion 11 or proximal portion. In correspondence of the second portion 12, or distal portion, the elongated body 13 presents a distributing element 14 for the sample of biological or microbiological material, which is configured for entering in use in contact with a substance, in particular a liquid or fluid substance and/or a sample of biological or microbiological material, and for allowing the distribution of this sample within the container 30.

In correspondence of the first portion 11, or proximal portion, the elongated body 13 comprises a connector 15 which is configured for allowing the removable coupling of the tool 10 with a supporting element, identified by the numeric reference 20, of a machine 40. In a particular embodiment, the connector 15 is specifically configured for removably coupling to a supporting element 20 of a pipetting machine, in particular to an automatic pipetting machine. This pipetting machine, according to a particular embodiment, comprises a (or is connected with a) electropneumatic device configured for allowing to collect a predetermined dose of fluid through a pipette, or tip, installed on the supporting element 20, and deposit this dose of fluid in a predetermined place, in particular a Petri dish as above described, following a process automated and made repeatable through a data processing unit. Some pipetting machines are configured for collecting, preferably through the actuation of a vacuum and of an air pressure, a predetermined amount of liquid typically comprised between 1 μl and 1 ml. Pipetting machines upon which the tool 10 can be installed can be of modular type, and in particular the supporting element 20 previously indicated can be the supporting element 20 of a module of a pipetting machine which has more modules arranged according to a predefined scheme.

Having a connector 15 configured as above described allows to use the machine 40 not only for pipetting operations, but also for operations of distribution of the sample of biological or microbiological material released by the pipette, or tip, using the movement capacities of the supporting element 20. This supporting element 20 can then be a supporting element 20 provided with a cavity connected to a pump, also adapted to be removably coupled to a pipette, or tip, (for example of plastic material) for pipetting machines, which is normally configured for being removably coupled with a pipette, or tip, and for exercising—through said cavity—an action of suction or of pressure inside the pipette, or tip, for respectively intaking or releasing a sample of biological or microbiological material. Therefore, thanks to the tool 10 object of the present disclosure is possible to use only one machine for carrying out two different processes: a first, pipetting, and a second, distribution of the sample of biological material.

The Applicant in particular points out that when the operations of pipetting and distribution of samples of biological or microbiological materials within containers 30 are simultaneously executed at high speed and/or on a great number of containers 30, the use of two different machines for carrying out the two processes above indicated becomes extremely expensive, and/or the manual movement of more containers 30 exposes to the risk of wrong movements and notably increases the processing times. For this reason, the use of a tool 10 for the distribution of samples of biological or microbiological material able to be installed on a machine typically adapted to carry out pipetting operations allows: a reduction of costs (with respect to the use of two different machines) and, at the same time, a reduction of processing times, in particular of times necessary to carry out said action of spreading or streaking of a plurality of samples of biological or microbiological material.

The removable coupling implies that in use, in particular after a single or multiple use on one or more samples, the tool 10 can be disconnected from the supporting element 20 of the machine 40 to be disposed of and replaced with another tool 10. In particular, a non-limiting embodiment of the tool 10 is of single use type, and is then realized to be used only one time before being disposed of: conveniently this embodiment presents at least the elongated body 13 which is realized in plastic material, in particular in an electrically or capacitively conductive plastic material or integrating a filament of electrically conductive metallic material. An alternative embodiment of the tool 10 is configured for being used more times, and is in particular configured for being sterilized a predetermined number of times through known technique (for example and in a non-limiting extent, through dry or moist heat, or UV or radiation with ionizing radiation and/or microwave radiation, and/or through chemical sterilization, for example by means of ethylene oxide or peracetic acid). When the tool 10 must be used more than one time, within the processing of samples of biological or microbiological material an attentive sterilization is important in order to prevent harmful phenomena of contamination of different samples. Conveniently, in this case, at least the elongated body 13 is realized in metallic material, preferably although non-limiting in Nickel-Chromium alloy, preferably and non-limiting with an amount of Nickel equal to or higher than 60% in weight, more preferably equal to or higher than 63% in weight or even more preferably equal to or higher than 65% in weight. For example, and in a non-limiting extent, at least it can be realized in UNS N06082 alloy (or equivalently Inconel 82 alloy), which is an alloy also comprising Manganese, Niobium and Iron, the latter in amount comprised between 0 and 3% in weight. The Applicant in particular notices that the use of this type of alloy is particularly convenient in order to allow the sterilization of the tool 10, because this alloy is highly resistant to high temperatures. The Nickel-Chromium alloy, and in particular the Nickel-Chromium Inconel 82 alloy presents a low thermal conductivity (specific thermal capacity). Thanks to this aspect, it is possible to reduce the heat transmission towards the first portion 11 or proximal portion, and then towards the machine 40, allowing therefore a better functioning and a lower risk of damage.

As a matter of fact, when the tool 10 is properly installed on the supporting element 20, the tool 10, and in particular at least the elongated body 13 and/or the distributing element 14, is subject to a procedure of high temperature sterilization; this process of high temperature sterilization takes place by radiation (in particular, by means of ionizing radiation) or by means of flame heating, and can reach temperatures also higher than 600° C., in particular higher than 700° C., and can reach temperatures until 800° C. Although the longitudinal extension of the tool 10 is small, the low conductivity of the above-described alloy allows therefore to transfer a very low amount of heat to the supporting element 20.

Preferably, although in a non-limiting extent, the elongated body 13 presents a determined flexibility degree and is at least partially, optionally entirely, flexible; this allows, in addition, to avoid damages on the movable parts of the machine 40 if by mistake the elongated body 13, and in particular the distributing element 14, enter in contact with a portion of the container 30.

The distributing element 14 is an electrically conductive element, and can be electrically and/or capacitively coupled with the elongated body 13. Thanks to this feature, the tool 10 in use acts as capacitive probe able to allow the detection of a contact which is realized between at least the distributing element 14 and the culture medium and/or the sample of biological or microbiological material. The wording “between at least” is herein used because according to the thickness of the culture medium, and/or to the assembly formed by the latter and by the sample of biological or microbiological material, the contact could take place not only with the distributing element 14 but also with at least part of the distal portion 12 of the elongated body 13.

As schematically shown in FIG. 3, in conditions of absence of contact with any substance, the tool 10 presents an its own capacity (herein defined as basic capacity) which can be read by a sensor positioned for example on the supporting element 20 or on the machine 40 itself. When the tool 10, in particular its distributing element 14 enters in contact with a substance (condition shown in FIG. 4), the capacity shown by the assembly formed by the tool 10 and by the substance itself is different with respect to the basic capacity (indicated in FIG. 3 with C₁), and this variation—which typically is sudden at the contact, as well as significant—represents a situation wherein the distributing element 14 is entered in contact with the substance. At least two operating conditions are identified in use:

a first operating condition of absence of contact between the distributing element 14 and the substance, wherein the basic capacity of the tool 10 is read; and

a second operating condition of presence of contact between the distributing element 14 and the substance, wherein the total capacity (indicated in FIG. 4 with C₂) resulting from the coupling between the distributing element 14 and the substance (and then, from the electrical and/or capacitive point of view, resulting from the coupling between the tool 10 and the substance) is varied with respect to the basic capacity.

In any case is foreseeable that at least a predetermined alteration threshold is defined with respect to the basic capacity for defining the switching between the first operating condition and the second operating condition. This switching between the first operating condition and the second operating condition determines the execution or the interruption of operations of movement of the supporting element 20 with respect to the container 30 which will be better described hereinafter.

Since the reading of a variation of a capacity imposes the realization of a kind of condenser, there is a dielectric between an electric supply source (aboard of the machine 40) and the distributing element 14. In order to reach the purposes of the present disclosure, the dielectric—and then the interruption of an electric continuity—can be present in principle both within the tool 10, and upwards of the supporting element 20 of the machine. For this reason, it is defined that the distributing element 14 is electrically or capacitively coupled to the elongated body 13. In an embodiment shown in the attached figures, there is electrical continuity along all the elongated body 13 and along the distributing element 14, and the latter is electrically conductive.

The Applicant has conceived different embodiments of the distributing element 14 which, even accomplishing at least the previously described function, are distinguished by various geometrical shapes and properties.

A first embodiment of the distributing element 14 is shown in FIG. 1 and in FIG. 2. In this first embodiment the distributing element 14 comprises a folded tubular portion such as to result misaligned with respect to the axis X along which the elongated body 13 substantially develops. As it is possible to observe from the same figures, the elongated body 13, with its second portion 12 or distal portion, reaches a first end height indicated with the reference Qe. A second end height is the one of the most external portion of the engaging portion 16. The first and the second end height define end heights within which the tool 10 is comprised in longitudinal direction. The curved portion of the distributing element 14 is not only misaligned with respect to the axis X but is also preferably folded backwards so that one end thereof is at a height, indicated in FIG. 2 with the reference Qi, which is more inner with respect to the first end height Qe.

In particular, in this embodiment, the distributing element 14 comprises a part of elongated body 13, which extends uninterruptedly into it or, equivalently is integral with the elongated body 13, representing in fact a folded part of the latter; the end of the distributing element 14, which is at said height Qi, is such as to be in substantial contact with the elongated body 13, in particular with an intermediate portion of said elongated body 13, and is joined to it through, preferably, a welding. In an alternative solution, this welding is not present, and the end of the distributing element is only in proximity of the elongated body 13. If there is the welding, it is realized a closed ring in substantial correspondence of the second portion 12 of the elongated body 13. In other words, the distributing element 14 therefore comprises a first end portion integral to the elongated body 13, in particular because extension or part of the latter, and a second end portion, different from the first end portion; the second end portion ends in position near the elongated body 13 (if there is no welding) or is rigidly joined, optionally welded, to the elongated body 13.

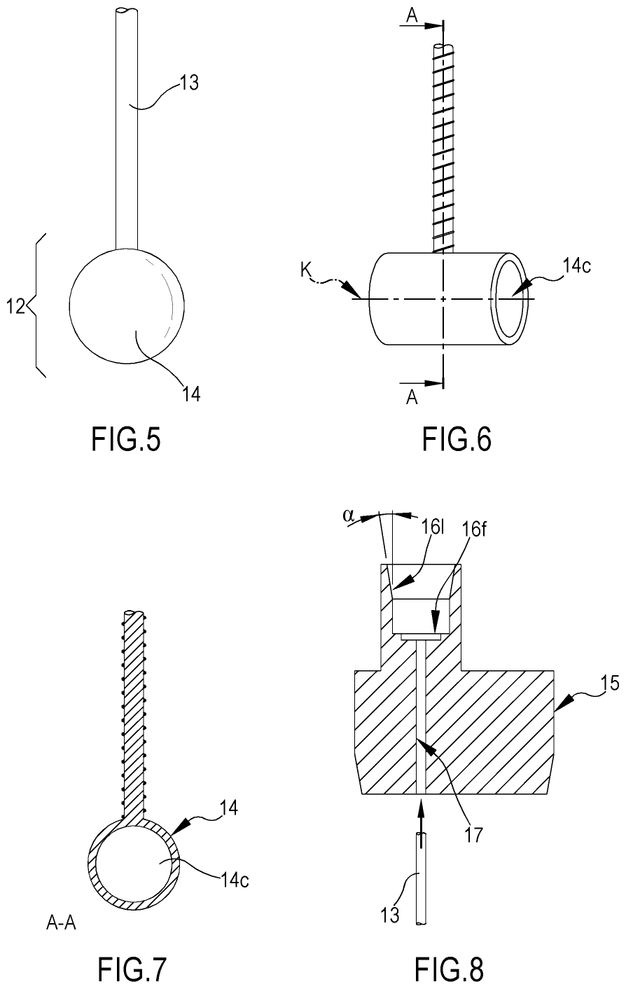

A second embodiment of the distributing element 14 is shown in FIG. 5. This second embodiment of the distributing element 14 presents a spheroidal body, in particular a spheroidal body whose diameter is higher with respect to the thickness that the elongated body 13 assumes along a direction substantially orthogonal with respect to the axis X, which is the axis along which the elongated body 13 substantially develops.

In an alternative embodiment, not shown in the attached figures, the distributing element 14 assumes a partially spheroidal shape, in particular hemispherical. A planar wall of the hemisphere can lay on a plane which comprises the axis X or which is defined on a pair of axes of which one is parallel to axis X, or alternatively can lay on a plane orthogonal with respect to the axis X.

A third embodiment of the distributing element 14 is shown in FIG. 6 (perspective view) and in FIG. 7 (sectional view, along lines A-A of FIG. 6). This third embodiment of the distributing element 14 comprises a hollow body, which detects a cavity 14c adapted at least to house a predetermined amount of substance, in particular a predetermined amount of fluid or liquid and/or sample of biological or microbiological material and/or a predetermined amount of culture medium. In the embodiment shown in the attached figures, the hollow body presents a substantially cylindrical shape with circular section and the cavity 14c of the hollow body detects an axis, indicated with the reference K, which is substantially orthogonal to the axis X. In a non-limiting embodiment, which is the one shown in FIG. 6 and in FIG. 7, the cavity 14c is passing from an end to another of the hollow body. The distributing element 14, by effect of the cavity, is not only able to distribute at least a sample of biological or microbiological material on a culture medium but, also, is able to contain and collect a predetermined amount, in particular a predetermined volume, of a substance, both solid or fluid. The Applicant in particular notices that the holding of a fluid substance, in particular substantially liquid, within the cavity of the distributing element 14 can take place also by effect of the surface tension of the fluid. In this case the hollow body can assume also shape of annular or toroidal element. The distributing element 14 of the present embodiment realizes therefore a calibrated loop adapted to collect substances, and the volume contained by it can be for example at least equal to 0.5 μl, or 1 μl, or 2 μl, or 5 μl, or 10 μl, or 30 μl.

The third embodiment is shown with an electrical conductor wrapped around the elongated body 13 which allows to bring electrical conduction from the distributing element 14 in proximity of the connector 15, in particular in correspondence of a contact portion that acts as electrode and that is better described hereinafter. The Applicant points out that the elongated body 13 can then be also electrically insulating, provided that it is ensured an electrical and/or capacitive coupling between the distributing element 14 and the contact portion or electrode previously mentioned. Therefore, in order to obtain the effect to make the variation of the capacity measurable, for the tool 10 is sufficient that there is an electrical and/or capacitive coupling between the distributing element 14 and the elongated body 13 or an electrical and/or capacitive coupling between the distributing element 14 and the connector 15, in particular in correspondence of the contact portion.

As a matter of fact, as it is possible to observe in FIG. 2, a part of the elongated body 13, in particular the first portion 11, or proximal portion, is introduced within the connector 15 and presents a contact portion 13s operatively accessible by an electrode 20a positioned on the supporting element 20; through the contact portion 13s the machine 40 previously mentioned can operate a reading of the variation of capacity. The contact portion 13s is then a portion electrically conductive too. The hidden position of the contact portion 13s allows to avoid false electrical couplings and preserves the latter from contaminations with dust or dirt or greases during the manipulation of the tool by operators. In a particular embodiment, the contact portion 13s is a contact portion integral with the elongated body 13.

As shown in FIG. 8, the connector 15 presents a body provided with a truncated cone-shaped engaging portion 16 which is configured for allowing the removable engagement with the supporting element 20. In the non-limiting embodiment shown in FIG. 8, the engaging portion 16 comprises a recess realized in the body of the connector 15 which comprises a side wall 16l and a bottom wall 16f ; preferably but in a non-limiting extent the side wall 16l has a cylindrical section, while the bottom wall 16f is substantially or at least partially flat and extends in particular along a plane orthogonal to an axis of the recess. In particular, the side wall 16l presents a first portion, in particular higher and nearer the entry of the recess, of truncated-cone section and a second portion, in particular laying deeper into the recess, of circular section. In an alternative embodiment, not shown, the side wall 16l can be realized in a single truncated-cone section. The conicity assumed by the side wall 16l with respect to the axis X is indicated in figure with the angle a. In an embodiment (not shown), however, the engaging portion 16 can comprise a portion which protrudes outside of the body of the connector 15.

The Applicant notices that between the second portion of the side wall 16l and the bottom wall 16f there is a step. This step causes the area of the bottom wall 16f to be lower with respect to the section of the second portion of the side wall 16l. This step allows to avoid that the supporting element 20 hits the bottom wall 16f.

The connector 15 presents also a guiding cavity 17 which presents an end which opens in correspondence of the bottom wall 16f of the engaging portion 16. The guiding cavity 17, which presents a substantially axial extension, is adapted to house at least part of the first portion 11 or proximal portion of the elongated body 13. When the tool 10 herein described is mounted, the elongated body 13 is introduced within the guiding cavity 17 and preferably, even though in a non-limiting extent, their reciprocal dimensions, in particular in direction transversal with respect to the axis X, are such that the introduction of the elongated body 13 takes place through insertion by contrast, i.e. with a sliding friction between the inner side surface of the guiding cavity 17 and the outer side surface of the first portion 11 or proximal portion of the elongated body 13. Subsequently it is realized a welding portion which, for example and in a non-limiting extent, can assume a substantially discoidal shape and which realizes the previously mentioned contact portion 13s, adapted in use to enter in contact with the electrode 20a. Alternatively, a welding portion can be a substantially punctiform portion and the welding can take place in substantial correspondence of the bottom wall 16f. When in discoidal shape, the shape of the contact portion 13s, and in particular its dimension transversally to the axis X are higher than the thickness of the elongated body 13; this also allows to avoid that the elongated body 13 can be extracted from the guiding cavity 17.

When the welding is of punctiform shape, the contact portion 13s is represented by the side wall 16l of the engaging portion 16; the Applicant has noticed that in this latter case the optimization of the electrical contact from the mechanical point of view, and then the reliability with which the variation of capacity can be determined, is also given by the truncated-cone shape of the engaging portion 16 and by the insertion with mechanical interference of the supporting element 20 in the recess of the engaging portion 16: the big side surface and the mechanical interference ensure an optimal electrical contact and/or a low contact resistance.

A process of assembling of the tool 10 comprises the holding of the connector 15, a subsequent introduction of part of the elongated body 13 within the guiding cavity 17 so that an end portion of the elongated body 13 is brought at least at a height substantially corresponding to the bottom wall 16f of the engaging portion and, subsequently, an execution (for example by means of an automatic machine, not described) of a welding in correspondence of said end portion so that said contact portion 13s is created laying upon, and in particular covering, at least in part (preferably substantially all) the bottom wall 16f. By laterally observing the connector 15, it is observed that both the guiding cavity 17 and the engaging portion 16 are in position not centred on the connector 15. In other words, on a determined plane which comprises the axis X, the body of the connector 15 assumes an asymmetrical shape. As it is possible to observe for example in FIG. 1, in FIG. 2 or in FIG. 8, the portion of the body of the connector 15 which is on the left of the axis X is less laterally developed with respect to the portion of the body of the connector 15 which is on the right of the axis X. The body of the connector 15 assumes a vaguely parallelepiped shape, the higher sides thereof present substantially planar walls and joined between them by semi-circular end portions. Thanks to the asymmetrical shape, the connector 15 can be arranged in seats 51 of a loader 50 or tray such that it is possible to define if the tool 10 has been positioned in correct or inverted way. This helps to allow the simplification of operations of automatic collecting of the tool 10 from a loader.

As it is possible to observe in FIG. 1, in substantial correspondence of the engaging portion 16, the connector 15 comprises a convex portion 15k which protrudes outside of the substantially planar side wall of the connector itself. This convex portion is present only on a side of the connector 15; on the other side, it is therefore absent. Conveniently, the Applicant has conceived a particular embodiment for the loader 50 or tray, wherein the seats 51 comprise a recess 51k of shape substantially duplicating in concave way the protrusion of the convex portion 15k. This helps even more the correct positioning of the tool 10.

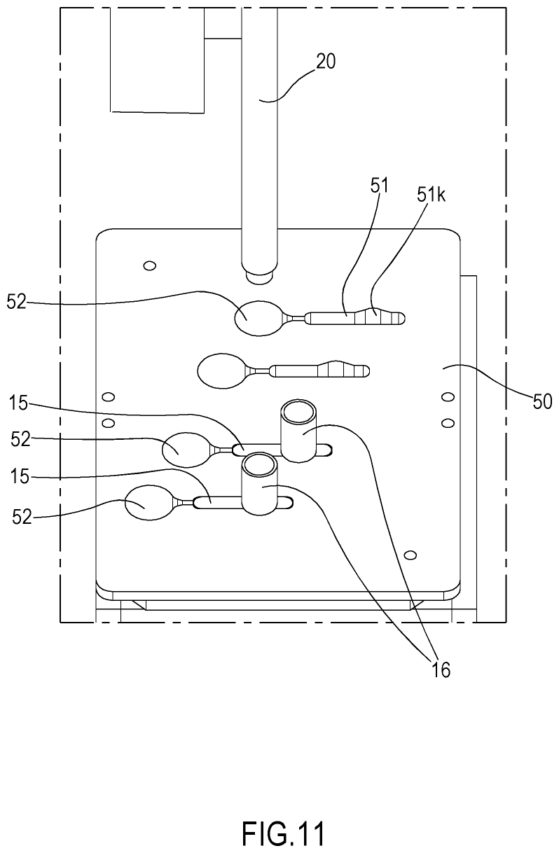

FIG. 11 shows seats which comprise a side portion with circular section, identified with the numeric reference 52, which is a side of a main portion of the seat 51 and is joined to it. This side portion 52 presents a diameter higher with respect to the transversal extension of the remaining portion of the seat. This side portion 52 of the seat 51 is conceived in order to allow a more agile and easier introduction of the distributing element 14. After the introduction of the distributing element 14 in the side portion 52, the tool 10 is translated along a direction orthogonal to axis X for introducing the connector 15 in the main portion of the seat.

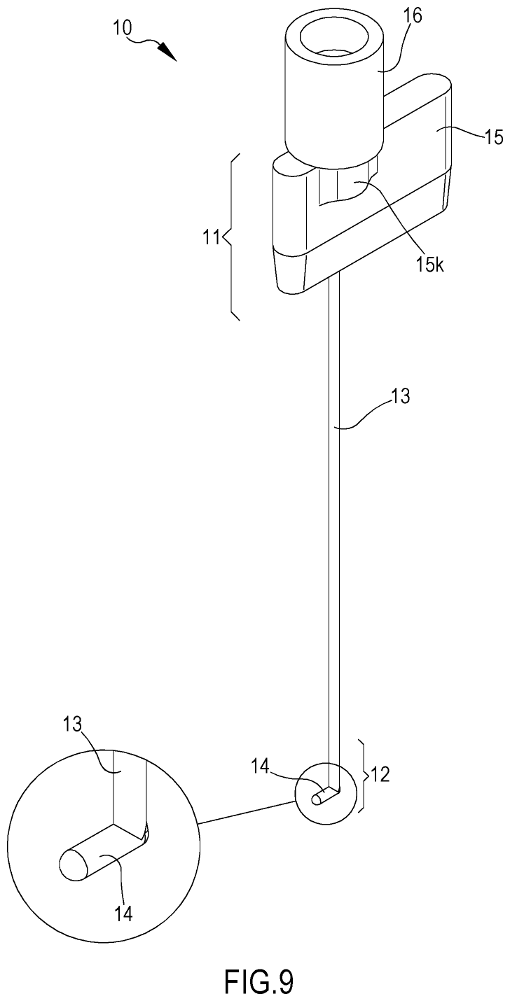

FIG. 9 shows another embodiment of the tool 10 object of the present disclosure. In this embodiment, the distributing element 14 has substantially a “L” shape and comprises a portion aligned along the axis X, and a second portion, joined to the first portion, which is instead aligned along a direction substantially orthogonal to axis X. Although in the embodiment shown in FIG. 9 the distributing element 14 is represented with a substantially circular section shape, in particular both in the first and in the second portion, other possible variants conceived by the Applicant comprise at least a second portion realized with a paddle shape, with a substantially laminar or planar configuration, in particular laying on a plane which comprises the axis X and an axis orthogonal to it or, alternatively, laying on a plane which comprises the axis orthogonal to axis X and an axis inclined orthogonal with respect to the axis X itself. This latter alternative can be defined as “Hockey stick” shape.

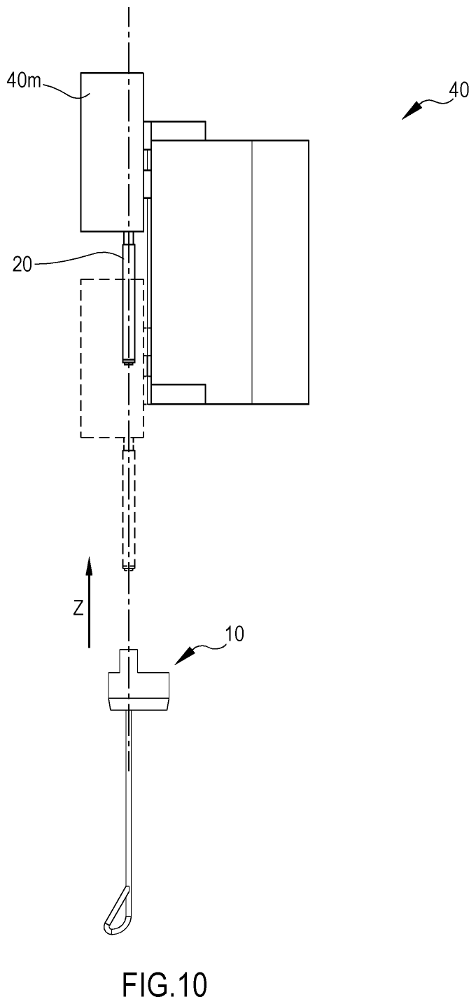

FIG. 10 schematically describes a side view of a machine, in particular a pipetting machine, which is in particular configured for allowing the removable coupling both of a pipette, or tip, and (alternatively to the pipette, or tip, itself) a tool 10 as the one object of the present disclosure. This machine, identified by the numeric reference 40, can comprise a module 40m movable at least axially along an axis Z, in use substantially vertical. The module 40m presents a supporting element 20 which in turn has an end portion which couples with the pipette, or tip, or alternatively with the tool 10.

Therefore the removable connection thus realized is such that to the supporting element can be alternatively connected tools, among which the pipette here described, or the tool 10 which forms object of the present disclosure.

When the tool 10 is appropriately coupled with the supporting element 20, the axis X of the elongated body 13 results parallel to the axis Z. In FIG. 10 in particular is possible to observe a configuration wherein a dotted version of the module 40m is in a position at a height considerably lower with respect to the height assumed by the module 40m represented with the continue line. The height at which there is the dotted version of the module 40m can be the height in correspondence of which the supporting element 20 enters in contact with the tool 10 (in particular, engages the tool 10) in the above-described modes.

FIG. 11 shows a schematic representation wherein it is possible to observe a module 40m in an operating configuration near the collecting of a tool 10 from a slot-shaped seat 51 of a loader 50 or tray; this loader 50, due to the predefined arrangement of seats 51, is such that it allows to position a plurality of tools 10 in a predefined spatial configuration.

It is described a processing sequence carried out on a container 30, in particular on a Petri dish, through a machine 40, in particular through a pipetting machine also configured for allowing the distribution of a sample of biological or microbiological material on a culture medium.

The machine 40 preferably comprises a data processing unit which in use executes a computer program which comprises portions of software codes which when executed cause the execution of at least part of the steps hereinafter described. This software program can be written in any programming language of known type. The data processing unit, or control unit, can be a processor of general-purpose type specifically configured for carrying out one or more parts of the process detected in the present disclosure through the software program or firmware, or be an ASIC or dedicated processor or an FPGA, specifically programmed for carrying out at least part of the operations of the process herein described. The memory support can be non-transitory and can be inside or outside of the processor, or control unit, or data processing unit. The machine 40 comprises also a memory support, or is operatively connected to a memory support, upon which is stored at least said computer program.

First of all, the container 30 is at first arranged on a support, for example a support having a plurality of arms 41 adapted to surround at least part of the container 30. This step can be manually carried out by an operator or through an automated process. The Applicant notices that in a non-limiting embodiment, the plurality of arms 41 comprises a triad of arms 41 substantially arranged at 120° the one with respect to the other; this embodiment is particularly efficient in supporting a container 30 of substantially circular shape, as is the container represented in the attached figures.

Alternatively, to the arms 41, or in combination with them, the support of the container 30 can comprise a suction cup, or an equivalent device, designed to retain the container 30 in correspondence of an its bottom wall by means of a pressure differential. The suction cup is particularly efficient when the bottom wall of the container 30 is planar and, in particular, smooth. In a particular embodiment of the machine 40, the suction cup is anyway installed on an actuator configured for allowing the rotation of the container 30, in particular around the axis X′.

Subsequently, the machine 40 is actuated for depositing a predefined amount, in particular a predefined volume, of a sample of biological or microbiological material within the container 30, wherein it is previously deposited a layer of culture medium. In details, for the depositing, the module 40m is moved preferably axially (along the axis Z), and with it is preferably axially translated the supporting element 20 (always along the axis Z), which houses a pipette, or tip, removably installed in correspondence of an its end. This actuation conveniently takes place through a movement command automatically given by the data processing unit.

The release of the predefined volume of the sample of biological or microbiological material by the pipette, or tip, is carried out through a removal of the vacuum previously exercised by the pump of the machine 40, or through air injection within the pipette, or tip, by means of an injection system, for example comprising a pump, of known type and therefore not described in details. In a preferred embodiment, the process of release of the predefined volume of sample of biological or microbiological material is carried out by approximating the distal end of the pipette, or tip, to the container 30.

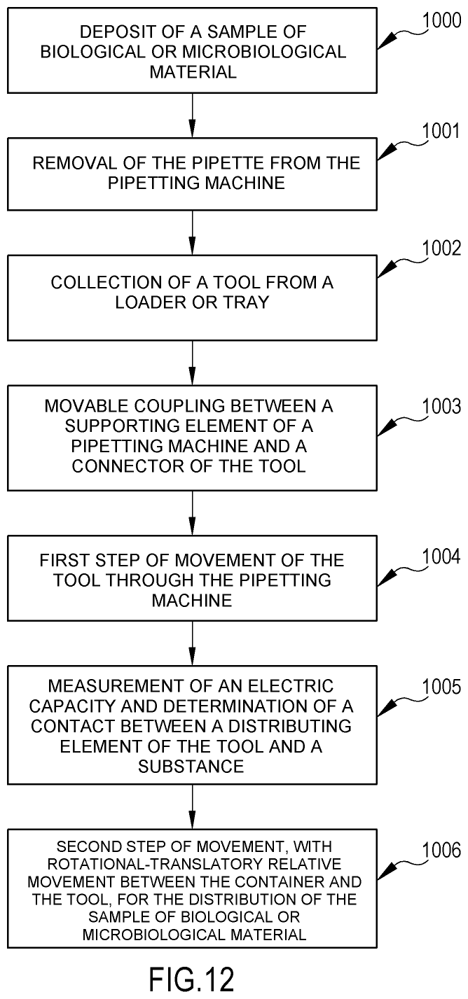

According to the present disclosure, the step herein described can be defined as a step (block 1000, diagram of FIG. 12) of depositing of a substance, in particular of a predetermined amount of fluid or liquid and/or sample of biological or microbiological material, in a container 30.

Once the release has been carried out, preferably even if non-limiting, the machine 40 is actuated so that the pipette, or tip, is spaced from the container 30, preferably through an axial movement of the module 40m and, consequently, of the supporting element 20 along the axis Z, so that the distal end of the pipette, or tip, is sufficiently spaced from the container 30.

Subsequently, it is executed a step of replacement of the pipette, or tip, wherein, or after which, the tool 10 is removably coupled to the machine 40. This step of replacement can advantageously be automatically carried out. In this step, in particular, the pipette, or tip, is removed from the end of the supporting element 20 for example through a manual technique and/or through a forced release ensured by movable portions installed on the machine 40, and—further subsequently—the data processing unit controls the machine 40 for carrying out a step of removable coupling (block 1003, diagram of FIG. 12), wherein the connector 15 of the tool 10 is removably coupled to the supporting element 20 of the machine 40.

In particular, the step of movable coupling 1003 is realized through an axial approximation of the distal end of the supporting element 20 with the connector 15, in particular with the engaging portion 16 and in particular comprises a subsequent introduction of the supporting element 20 at least partially within the engaging portion 16 such as a sliding friction and a contrast force are exercised on the truncated-cone shaped side wall 16l of the latter, sufficient to retain the tool 10 on the supporting element 20 at the uplifting of the tool 10 itself. In a non-limiting embodiment, as the introduction of the supporting element 20 within the engaging portion 16 increases, due to the conicity assumed by the side wall 16l, said sliding friction and/or said contrast force increases with the progressive introduction.

The Applicant notices that in the step of movable coupling 1003 is realized also an electric and/or capacitive coupling between the electrode 20a present on the supporting element 20 and the contact portion 13s; it is important that this step of removable coupling 1003 determines this electric and/or capacitive coupling in order to determine the possibility to detect the contact of a substance, in particular a sample of biological or microbiological material previously deposited in the container 30 at the contact of the latter at least through the distributing element 14.

The step of movable coupling herein described is a step which can lay within a step of collecting (block 1002, diagram of FIG. 12) wherein a tool 10 is specifically and previously arranged in an appropriate seat 51 of a loader 50 or tray adapted to contain a plurality of tools. In this case the relative position between the module 40m and/or the supporting element 20 and the tool 10 is known at least to the data processing unit, and therefore the movement at least of the module 40m can be previously programmed and carried out in routine by means of the software program previously described. The module 40m, and therefore the supporting element 20 are translated along a substantially horizontal plane, in particular keeping the axis of the supporting element 20 always substantially vertical, between a first position of starting and a position of arrival coinciding with the position wherein, axially along a vertical direction, there is the engaging portion 16 of the connector 15. Subsequently, the module 40m is moved to be lowered to the height sufficient to cause the insertion of the distal portion of the supporting element 20 within the engaging portion 16 as above-described. For this reason, it is possible to observe that the step of movable coupling comprises a composite movement of the supporting element 20, and then of the module 40m that supports it, along substantially a first axis substantially horizontal (axial approximation to the engaging portion 16) and, subsequently, along a second vertical axis and substantially orthogonal to the first axis (approximation and introduction of the distal portion of the supporting element 20 within the engaging portion 16).

The step of collecting 1002, in particular, comprises the uplifting of at least a tool 10 at a height sufficient also for the distal end of the tool 10 to be at a higher height, or anyway not in interference, with the loader 50 or tray.

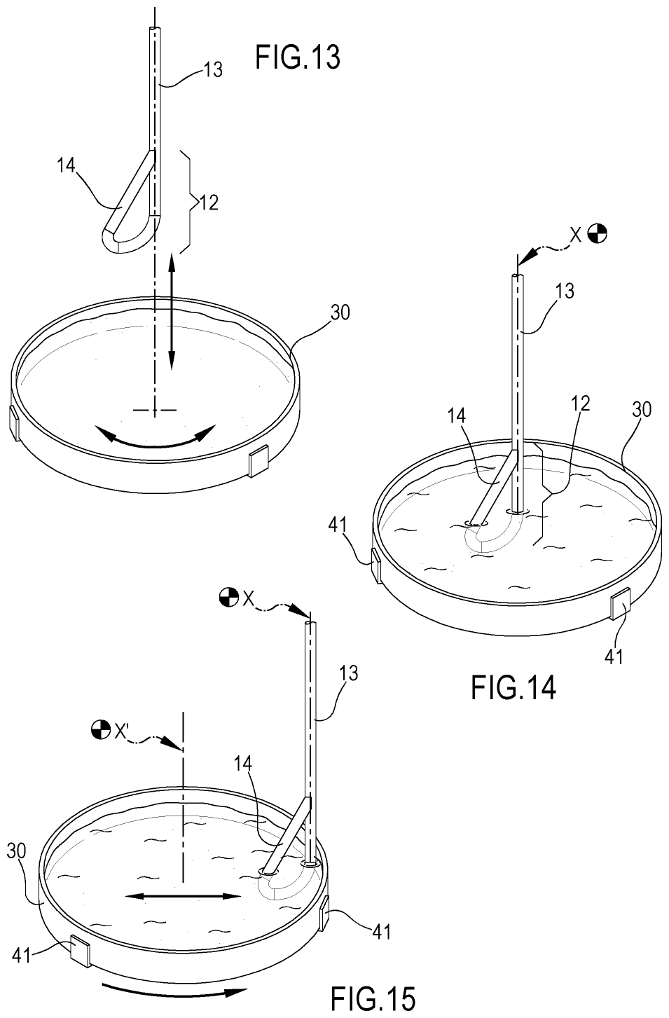

At this point a first step of movement (block 1004, FIG. 12) of the tool 10 with respect to the container 30 takes place, which is schematically represented in FIG. 13; in this first step of movement 1004, the tool 10 is moved, preferably axially so that its distributing element 14 moves between a first and more remote position with respect to the container 30 and a second position, wherein it is in a position nearer to the container 30.

In particular, the second position is a position wherein the distributing element 14 is in substantial contact with the substance S contained in the container 30, and in particular is in substantial contact with the sample of biological or microbiological material. Preferably but in a non-limiting extent, during the first step of movement 1004 the container 30 is kept in a fixed position.

In the continuation of the first step of movement 1004, the machine 40 carries out a measurement of a capacity (block 1005, FIG. 12) by means of the tool 10, in order to determine when the distributing element 14 enters in contact with a substance S. In particular, in an embodiment, the measurement of the capacity is carried out uninterruptedly during the first step of movement 1004. The first step of movement 1004 comprises a linear translation of the module 40m along the axis Z, which ends when it is detected a variation of capacity measured through the tool 10 and the distributing element 14 higher than the predetermined fixed threshold.

In a particular embodiment, the distributing element 14, after the variation of capacity, can be further introduced within the sample of biological or microbiological material and/or within the culture medium present in the container 30. This introduction takes place through a translation along the Z axis, and this translation—which preferably is comprised between 1/10 mm and 1 mm included ends, takes place uninterruptedly in the first step of movement 1004 or in an intermediate step of insertion which takes place after the first step of movement 1004. Therefore, in this embodiment, the second position is a position wherein at least part of the distributing element 14 is introduced within the sample of biological or microbiological material and/or within the culture medium contained in the container 30.

At this point, the process comprises a second step of movement (block 1006, FIG. 12) of the tool 10. In this second step of movement, which is schematically represented in FIGS. 13 and 14, the machine 40 actuates a particular relative movement between the tool 10 and the container 30.

In details the second step of movement is the step specifically designed to cause a distribution, optionally a substantially uniform distribution, of the sample of biological or microbiological material on the culture medium arranged within the container 30. The second step of movement 1006 comprises a relative movement between the tool 10 and the container 30 within which the distributing element 14, in particular a portion near the height Qe, remains in substantial contact with the substance placed in the container 30, and i.e., with the sample of biological or microbiological material and/or with the culture medium. By effect of the relative movement between the distributing element 14 and the container 30, this distributing element 14 traces a predetermined trajectory on the area enclosed by container 30.

As it is schematically represented in FIG. 15, the relative movement comprises a composite movement wherein the container 30 is moved around an axis X′ substantially parallel to the axis X, along which the elongated body 13 develops, and also comprises a translation of the elongated body 13, and consequently of the distributing element 14, along a plane which comprises the axis X. It is therefore a translation of the elongated body 13 wherein the latter is kept substantially in vertical direction; considering the axis X′ as the axis upon which is centred the container 30, with said translation the distance between the axis X′ and the axis X is varied. In particular, the translation of the elongated body 13 and the rotation of the container 30 along the axis X′ take place simultaneously. This implies that the machine 40, in the second step of movement, is configured for simultaneously actuating a first motor adapted to control the rotation of the container 30 and a second motor adapted to control the translation of the elongated body 13.

This simultaneous movement should not be understood in a limiting way. The Applicant points out that the action of spreading or streaking that the tool 10 and the machine 40 are configured for executing differ in the following characteristics.

The “spreading” is a technique wherein a container for samples of biological or microbiological material is simultaneously rotated with a movement of the tool 10, which carries out a linear translation as above-described.

The “streaking” is a technique wherein a container for samples of biological or microbiological material is temporarily kept still in a predetermined position, while the tool 10 is moved within the area of the container itself, for example and in particular a first sub-portion substantially peripherical of said area, and wherein after the movement, the tool 10 is stopped and the container for samples of biological or microbiological material is rotated for a predetermined angle. The rotation is stopped, and at this point the tool 10 is again moved within the area of the container, in a second sub-portion contiguous to the previous one. Therefore, in an alternative embodiment, the rotation of the container 30 and the movement of the tool 10 can take place in a temporally alternated way. In particular, the seeding of the sample of biological or microbiological material can take place on a predefined number of quadrants, for example three or four quadrants. The angle of rotation of the container 30 is associated to, and/or depending upon, the number of quadrants of the seeding.

Preferably but in a non-limiting extent, the vertical plane along which the elongated body 13 is moved, and upon which lays the axis X, in the second step of movement is always the same. This simplifies the electronic control of the machine 40.

The Applicant notices that the angular speed of rotation of the container 30 must not be excessive, in order to avoid that by effect of the centrifugal force induced by the rotation, the culture medium and/or the sample of biological or microbiological material are arranged in substantial correspondence of a retaining side wall of the container 30, reducing the amount present in correspondence of more central portions of the container itself.

The Applicant notices that the rotation of the container 30 along an axis X′ orthogonal to a horizontal plane allows, in a preferred and non-limiting embodiment, not to have the need of continuing the execution of the step of electronic measurement of the capacity 1005 during the continuation of the second step of movement.

At the end of the second step of movement 1006, the distributing element 14 is uplifted through a linear translation along a direction parallel to the axis X for a length sufficient to remove the substantial contact with the culture medium and/or with the sample of biological and/or microbiological material. In this final step, therefore, the supporting element 20 is again moved along the axis Z. In this way the container 30 can be removed by the arms 41 for being made subject of other manipulations.

It has been previously indicated that the program for computer is configured for actuating the machine 40 in order to carry out one or more steps of the previously described procedure. The Applicant in particular notices that a particular embodiment of the computer program comprises portions of software code which when executed cause the execution of a first step of movement 1004 of the movable supporting element 20 of the machine 40, the supporting element 20 being configured for being removably connected with the tool 10 herein described. Through the first step of movement 1004 of the supporting element 20, the distributing element 14 of the tool 10 for the distribution of a sample of biological or microbiological material is moved between:
a first position, wherein the distributing element 14 is in a position remote with respect to the container 30, and
a second position wherein the distributing element 14 is in a position at least nearer to the container 30, and in particular in a position in substantial contact with a substance contained in the container 30.

The computer program also causes the execution of a step of electric or electronic measurement of a capacity 1005 detected through the tool 10: in the step of measurement of the capacity 1005, carried out at least partially simultaneously to the first step of movement 1004 of the tool 10, the machine 40 carries out a measurement of a capacity through an electrical and/or capacitive contact with an electrically conductive portion of the tool 10 and wherein, when said machine 40 measures a variation of capacity, the movement of the supporting element 20 in the first step of movement 1004 is at least temporarily stopped so that the distributing element 14 is in substantial contact with the sample of biological or microbiological material and/or with the predetermined amount of culture medium; optionally, the computer program is configured for causing another movement in order to introduce partially the distributing element 14 within the substance contained in the container 30.

The computer program also comprises a step of distribution of the sample of biological or microbiological material by means of the tool 10, executed after the step of measurement of the capacity 1005. In particular, the computer program can be configured for simultaneously moving the container 30 and the distributing element 14 or, alternatively, for moving the container 30 and the distributing element 14 in an alternated way, so that to realize a personalized and/or individualized pattern of distribution of the sample of biological or microbiological material.

Another and non-limiting embodiment of the computer program initially causes the execution of an actuation of the pipetting machine 40 for the execution of a step 1000 of depositing of a sample of biological or microbiological material, through a tip or pipette of a pipetting machine, within a container 30 provided with a culture medium. Subsequently it causes an actuation of the pipetting machine 40 for the execution of a step of replacement, in particular automatic, of the tip or pipette with the tool 10 for the distribution of a sample of biological or microbiological material, wherein the tool 10 is coupled to the pipetting machine 40 instead of the tip or pipette (block 1001); finally the program causes an actuation of the pipetting machine for the execution of a step of distribution of the sample of biological or microbiological material on the culture medium through the tool 10 moved by the pipetting machine.

The invention is not limited to the embodiments represented in the attached figures; for this reason, the reference numbers indicated in the claims must not be considered as limiting, being introduced only with the purpose of increasing the intelligibility of the claims themselves.

It is finally clear that to the object of the present disclosure can be applied additions, modifications or variants obvious for the expert in the art, without escaping from the scope given by the attached claims.

1. Tool (10) for the distribution of a sample of biological or microbiological material, the tool (10) comprising:
an elongated body (13), provided with a first portion (11), or proximal portion, and a second portion (12), or distal portion, different from the first portion (11);
a distributing element (14), positioned in correspondence of the second portion (12), the distributing element (14) being configured at least for distributing a sample of biological or microbiological material on a culture medium;
a connector (15), positioned in correspondence of the first portion (11), the connector (15) being configured for, and/or specifically designed to, allowing the joining of the tool (10) with a supporting element (20) of a machine (40) configured for carrying out a distribution of a sample of biological or microbiological material, in particular of a pipetting machine (40) configured for carrying out a distribution of a sample of biological or microbiological material;
wherein the connector (15) is adapted and specifically configured for being removably coupled to a supporting element (20) in alternative to another tool, in particular to a tip or pipette for the collection and/or deposit of the sample of biological or microbiological material.

2. Tool according to claim 1, wherein the distributing element (14) is electrically and/or capacitively coupled to the elongated body (13) and/or to the connector (15); the tool (10) being configured for acting, in use, as capacitive probe adapted to allow the detection of a contact at least of the distributing element (14) with a substance, in particular with the culture medium and/or with the sample of biological or microbiological material and for allowing to keep said contact during the movement of the tool for the distribution of the sample of biological or microbiological material.

3. Tool according to one or more of the preceding claims,
wherein at least part of the elongated body (13), in particular the first portion (11), is introduced into the connector (15) and presents a contact portion (13s) operatively accessible by an electrode (20a) positioned on the supporting element (20) of the machine (40),
wherein the contact portion (13s) is an electrically conductive portion,
wherein the contact portion (13s) is configured for allowing in use to detect a capacity difference, and/or for transmitting an electric signal of capacity variation toward the electrode (20a), at the contact at least of the distributing element (14) with the substance, in particular with the culture medium and/or with the sample of biological or microbiological material.

4. Tool according to claim 3, wherein:
the connector (15) comprises an engaging portion (16) configured for allowing the coupling of the connector (15) with the supporting element (20) and wherein the engaging portion (16) comprises a recess adapted to house at least part of the supporting element (20),
the contact portion (13s) is realized in correspondence of the engaging portion (16), in particular within the recess of the engaging portion (16);
the contact portion (13s) is in electrical contact with the elongated body (13),
and wherein the elongated body (13) is realized in electrically conductive material.

5. Tool according to one or more of the preceding claims, wherein:
the elongated body (13) substantially and/or mainly extends along an axial direction (X);
the elongated body (13) is at least partially flexible, optionally integrally flexible;
the connector (15) is fixed in a non-removable and/or substantially rigid way to the elongated body (13),
the elongated body (13) presents at least a central portion, interposed between the first portion (11) or proximity portion and the second portion (12) or distal portion, of substantially cylindrical shape.

6. Tool according to one or more of the preceding claims, wherein:
the distributing element (14) presents an asymmetric shape,
the distributing element (14) is integral with the elongated body (13), and wherein the distributing element (14) comprises a substantially electrically and/or capacitively conductive tubular portion; the substantially tubular portion being folded such as to misalign itself from the axis (X) along which the elongated body (13) substantially develops.

7. Tool according to claim 6, wherein the distributing element (14) presents a substantially hooked shape and/or comprises a first end portion directly joined to the elongated body (13) and a second end portion, different from the first end portion and juxtaposed to the first end portion; the second end portion ending in a position proximal to the elongated body (13) or being rigidly joined, optionally welded, to the elongated body (13).

8. Tool according to one or more of claims 1-5,
wherein the distributing element (14) comprises a hollow body, which defines a cavity (14c) adapted at least to contain a predetermined amount of substance, in particular a predetermined amount of fluid or liquid and/or sample of biological or microbiological material and/or a predetermined amount of culture medium,
wherein the cavity (14c) has a circular section, or a rectangular or square section, and has an its own axis (K) oriented in a direction which is transversal, optionally substantially orthogonal, to the axis (X) along which the elongated body (13) substantially develops,
and wherein the cavity (14c) is adapted to contain a substance volume at least equal to 0.5 μl, or 1 μl, or 2 μl, or 5 μl, or 10 μl, or 30 μl, and/or wherein the cavity (14c) is adapted to contain a predetermined amount of fluid or liquid by surface tension.

9. Tool according to one or more of the preceding claims, wherein the tool (10) is a tool adapted to, and/or specifically designed to, being used a plurality of times, and in particular is a sterilisable tool (10), and wherein at least the elongated body (13) is realized at least partially in metallic material, in particular in a metallic material with low thermal conductivity.

10. Process of distribution of a sample of biological or microbiological material on a culture medium, the process comprising:
a step (1000) of depositing of a sample of biological or microbiological material, through a tip or pipette of a pipetting machine (40), within a container (30) provided with a culture medium;
a step of replacement of the tip or pipette of the pipetting machine (40) with a tool (10) for distributing a sample of biological or microbiological material according to one or more of the preceding claims, wherein the tool (10) is coupled to the pipetting machine (40) instead of the tip or pipette;
a step of distributing of the sample of biological or microbiological material on the culture medium through the tool (10) moved by the pipetting machine (40).

11. Process according to claim 10, wherein the step of replacement of the tip or pipette of the pipetting machine (40) with the tool (10) comprises a step of removable coupling (1003), wherein a connector (15) of the tool (10), is removably coupled to the supporting element (20) of the pipetting machine (40); and wherein the process further comprises a first step of movement (1004) of the tool (10) wherein the distributing element (14) is moved between a first position, wherein the distributing element (14) has been coupled to the pipetting machine (40) and is in a remote position with respect to the container (30), and a second position wherein the distributing element (14) is in a position closer to the container (30) and in contact at least with the sample of biological or microbiological material;
and wherein after the first step of movement (1004) is stopped, a step of distributing of the sample of biological or microbiological material by means of the tool (10) takes place.

12. Process according to claim 10 or claim 11, comprising a step of electronic measurement of a capacity (1005), wherein said capacity is detected through the tool (10), and wherein the step of measurement of the capacity (1005) is carried out at least partially simultaneously with the first step of movement (1004) of the tool (10),
wherein, after the detection of a capacity variation indicating a contact between the distributing element (14) and the sample of biological or microbiological material, the first step of movement (1004) ends and begins the step of distributing of the sample and/or wherein in the first step of movement (1004) the container (30) is kept in a predetermined position, in particular in a fixed position.

13. Process according to one or more of claims 10-12, wherein the step of removable coupling (1003) comprises an electric and/or capacitive coupling realized between an electrode (20a) positioned on the supporting element (20) and a contact portion (13s) of the elongated body (13);
the electric and/or capacitive coupling being such that, through the electrode (20a), the machine (40) can detect when the distributing element (14) enters in substantial contact at least with the sample of biological or microbiological material and/or with the culture medium.

14. Process according to one or more of claims 10-13, wherein the step of distributing of the sample of biological or microbiological material by means of the tool (10) comprises a second step of movement (1006), wherein a relative movement between the distributing element (14) and the container (30) takes place such that, in the continuation of the relative movement, the distributing element (14) remains in substantial contact with the sample of biological or microbiological material and/or with the culture medium contained in the container (30) and wherein, through the relative movement, the distributing element (14) tracks a predetermined trajectory on the area enclosed by the container (30).

15. Process according to one or more of the preceding claims 10-14, wherein:
the step of distributing of the sample of biological or microbiological material by means of the tool (10), or the second step of movement (1006), comprises a rotation of the container (30) around an axis (X′) substantially parallel to the axis (X) along which the elongated body (13) substantially develops; and/or
the step of distributing of the sample of biological or microbiological material by means of the tool (10), or the second step of movement (1006) comprises a translation of the elongated body (13) along a plane comprising the axis (X) along which the elongated body (13) substantially develops and/or comprises a translation of the body (13) with respect to the area enclosed by the container
in the step of distributing of the sample of biological or microbiological material by means of the tool (10), or in the second step of movement (1006), the rotation of the container (30) and the translation of the elongated body (13) are carried out at least partially simultaneously or are carried out in alternated way; and/or wherein the container (30) is a container configured for containing samples of biological or microbiological material, the container (30) preferably comprising a Petri dish.
